(19) United States
(12) Patent Application Publication
(10) Pub. No.: US 2010/0203156 A1
(43) Pub. Date: Aug. 12, 2010

(54) XENON AS A TREATMENT FOR HEMOGLOBINOPATHY

(76) Inventor: Steffen E. Meiler, Evans, GA (US)

Correspondence Address: BRETT BARTEL, 2649 OLD NORCROSS RD, TUCKER, GA, 30084, US

(21) Appl. No.: 12/703,184

(22) Filed: Feb. 9, 2010

Related U.S. Application Data
(60) Provisional application No. 61/207,133, filed on Feb. 9, 2009.

(51) Int. Cl. A61K 33/00; A61P 25/00; A61P 7/00; A61P 7/06

(52) U.S. Cl. 424/600

(57) ABSTRACT

The method disclosed herein entails treating a person, with sickle cell disease and who is suffering a pain crisis, with an effective amount of xenon, which alleviates the pain. Inhalation of xenon at subanesthetic concentrations has significant analgesic properties, which are mechanistically independent from the opioid receptor system. Inhaled xenon diminishes the propensity of sickle hemoglobin to aggregate by occupying critical internal cavities within the sickle hemoglobin molecule. Decreased hemoglobin polymerization and red blood cell sickling is beneficial during acute crisis and reduces the high incidence of sickle cell related complications after surgery.

### CROSS REFERENCE TO RELATED APPLICATIONS

This application claims priority to U.S. Ser. No. 61/207,133, entitled “Xenon as a Treatment for Hemoglobinopathy” to Steffen Meiler filed Feb. 09, 2009.

### BACKGROUND

1. Field of the Invention

This invention is generally in the field of treating pain for people with sickle cell disease. The invention provides a method for using xenon gas for the treatment of pain during a pain crisis for a person with sickle cell disease.

2. Background of the Invention

Sickle cell disease (“SCD”). SCD is a devastating, heritable disorder of hemoglobin that affects ˜100,000 patients in the U.S. and several million patients worldwide. The genetically defective hemoglobin (β6 Glu Val) demonstrates a strong physical bias to form insoluble aggregates at low oxygen tensions inside of circulating red blood cells (RBCs). These sickle hemoglobin (HbS) aggregates significantly alter the normal shape and function of the affected RBCs, resulting in distorted and rigid cells that tend to hemolyze, adhere to vascular endothelium, or passively obstruct small blood vessels in vital organs. The formation of insoluble hemoglobin is the initiating event that leads to a complex clinical syndrome characterized by chronic hemolysis, acute painful vaso-occlusive crisis, and chronic organ damage.

Unfortunately, there is no cure for sickle cell disease. The most common symptom of sickle cell disease is pain. During a painful crisis, pain may seem to come from the bones, usually in the arms, hands, legs, feet, or back. Also, there may be pain in the stomach or abdomen, or in the chest.

The terrible pain of a sickle cell crisis often requires a trip to the emergency room. Some health care professionals question the pain of sickle cell patients and administer insufficient narcotic drugs, which are presently the only effective treatment for this type of pain. Other sickle cell patients are relatively refractory to the effect of narcotics because they take these drugs regularly to manage their chronic pain. This leaves these patients with few to no other treatment options during the height of their acute painful crisis. This has led to a number of bad experiences in emergency rooms and hospitals and has undermined the trust of sickle cell patients into their health care professionals and institutions. It is important that patients are able to find treatments that do not lead to addictions and that allow doctors to take good care of them.

There are drugs used in the treatment of pain which are known in the literature and to the skilled artisan. For many patients opioid medications (also called narcotics) can be extremely helpful, particularly during a painful crisis. However, patients may worry about addiction to pain medications, particularly opioids. Patients who have many crises can sometimes benefit from taking opioid medications daily, along with additional pain medication during crises. The daily opioids can help reduce the number of crises and make the pain less severe.

Other medications that may be prescribed by a doctor for relief from sickle cell pain are: hydroxyurea, anti-inflammatory medications, such as ibuprofen, steroids, and certain antidepressant medications or anticonvulsant medications also may help relieve pain.

There remains a need for better treatment of pain for sickle cell patients that is effective in treating the pain without the risk of addiction and other related side-effects.

It is therefore an object of the present invention to provide a method for use in alleviating the pain from sickle cell patients suffering a pain crisis.

### SUMMARY OF THE INVENTION

This disclosure is proposing two novel indications for the use of inhaled xenon in patients with sickle cell disease.

The physical and clinical characteristics of xenon are such that controlled inhalation of this noble gas provides a novel therapy for sickle cell patients in acute painful crisis and serves as a superior anesthetic agent during surgery. This invention describes that inhaled xenon diminishes the propensity of sickle hemoglobin to aggregate by occupying critical internal cavities within the sickle hemoglobin molecule. Decreased hemoglobin polymerization and red blood cell sickling is beneficial during acute crisis and reduces the high incidence of sickle cell related complications after surgery. Inhaled xenon also provides a novel method of pain control during painful crisis. Inhaled xenon is also beneficial to sickle cell patients undergoing surgery. Inhalation of xenon at subanesthetic concentrations has significant analgesic properties, which are mechanistically independent from the opioid receptor system. This feature is critical for the proposed indication and provides significant benefit to the sickle cell patient.

### DETAILED DESCRIPTION OF THE INVENTION

In a broad aspect, the present invention relates to the use of xenon for relieving pain associated with hemoglobinopathy.

Xenon: Physical and Chemical Characteristics.

The noble gas xenon (atomic number 54) is a trace gas in Earth's atmosphere and is manufactured by fractional distillation of liquefied air. Xenon is a member of the zero-valence elements that are called noble or inert gases. It is inert to most common chemical reactions (such as combustion, for example) because the outer valence shell is complete with eight electrons. This produces a stable, minimum energy configuration in which the outer electrons are tightly bound, making it unlikely to form covalent bonds. Xenon has a low ionization potential, allowing its electron shell to be polarized by surrounding molecules. This induces a dipole that makes xenon capable of biological interactions, including the binding to proteins. Xenon is a colorless, odorless, non-flammable, non-explosive gas that is free of occupational and environmental concerns. Xenon is approved for human use as an anesthetic agent in ten European countries and other parts of the world. Studies have so far failed to reveal any toxic, allergic, mutagenic, fetotoxic or carcinogenic properties. In contrast to other potent anaesthetics, xenon is odorless, easier to titrate, is better tolerated, and provides superior pain relief.

Thus, the present invention relates to the use of xenon to control the pain associated with a pain crisis in sickle cell patients.

Inhaled Xenon: Preventing Aggregation of Sickle Hemoglobin (HbS).

Inhaled xenon diminishes the propensity of sickle hemoglobin to aggregate by occupying critical internal cavities within the sickle hemoglobin molecule. Decreased hemoglobin polymerization and red blood cell sickling is beneficial during acute crisis and reduces the high incidence of sickle cell related complications after surgery.

Adult hemoglobin (HbA) is a tetrameric molecule that consists of two α- and two β-protein subunits bound to a non-protein heme group. The gene defect in SCD is a single nucleotide A to T transversion of the β-globin gene, which results in glutamate to be substituted by valine at position 6. This mutation in sickle hemoglobin (HbS) causes no apparent effect on the secondary, tertiary, or quaternary structure of oxy-HbS, but in the deoxy state it exposes a hydrophobic patch on the protein between the E and F helices. The hydrophobic residues of the valine at position 6 of the beta chain are able to associate with the hydrophobic patch of neighboring deoxy-hemoglobin molecules, causing HbS to aggregate and to form fibrous precipitates. Xenon binds avidly to hemoglobin. It is estimated that during xenon inhalation 45% of the xenon in blood is carried by hemoglobin. Henry's law is observed, which states that at a constant temperature, the amount of a given gas dissolved is directly proportional to the partial pressure of that gas. The amount of xenon bound by reduced and oxyhemoglobin is approximately the same. The xenon association rates for hemoglobin are 1-2 times faster than those for O2 (Kon[M-1S-1]: Xe: 6×107 O2: 3.3×107) but are 10 times faster than those for CO (Kon[M-1S-1]: CO: 4.6×106). X-ray diffraction analysis showed that xenon occupies internal cavities within the hemoglobin molecule. Xenon binds to one distinct site inside each of the α- and β-subunits, but close (6 Å) to their external surface between the AB and the GH corners of the Hb molecule.

Based on this structural information, xenon binding will change the surface configuration of sickle hemoglobin at critical locations to interfere with the aggregation of HbS monofilaments into long straight rods. HbS rods consist of six to eight hemoglobin monofilaments which are wound around the tubular surface with a helical pitch of about 3000 Å. Other gases, such as propane, ethane, and methane, which are not in clinical use but have a compact shape and a polarizability similar to that of xenon, have previously been shown to prevent and reverse HbS aggregation.

Inhaled Xenon: Analgesic Properties.

In a preferred embodiment of the invention, an effective amount of xenon is administered to a patient in pain. The administration of xenon is through inhalation. The xenon is inhaled with a mixture of oxygen or otherwise breathable gases. Controlled inhalation of xenon provides a therapy for sickle cell patients in acute painful crisis.

A further embodiment of the invention, provides treatment of xenon where the effective amount of xenon is 10%-20% xenon inhaled with oxygen.

It is important to emphasize that the herein disclosed indications for inhaled xenon are achieved at different doses. Xenon will be delivered at a concentration of 10-20% inhaled with oxygen during acute crisis. At this dose patients will maintain consciousness and alertness and are expected to be free of nausea and vomiting, which has been observed at higher inhaled concentrations of xenon (>30%). Inhaled xenon at a dose of ˜20% has been shown in healthy volunteers to significantly decrease the response to various experimentally induced and well standardized forms of pain. The analgesic potency of xenon is comparable or superior to nitrous oxide and produced analgesia to ischemic, electrical, mechanical, and heat stimulation, but not to cold pain. The ability of xenon to diminish the perception to experimentally induced ischemic pain is particularly relevant because pain during vaso-occlusive crisis is predominantly ischemic in origin. In animal studies, xenon directly inhibited the nociceptive responsiveness of spinal dorsal horn neurons and therefore, unlike nitrous oxide, did not require the involvement of the descending inhibitory system. Furthermore, Xenon has been shown to reduce nociception at various ages, unlike nitrous oxide, which is ineffective in the young. The antinociceptive properties of Xenon are independent of opioidergic or adrenergic receptors, which enhances Xenon's attractiveness as a therapeutic agent for crisis pain.

Inhaled Xenon: A General Anesthetic Agent for SCD.

In one embodiment of the invention, an effective amount of xenon is administered to a sickle cell patient suffering a pain crisis. In another embodiment xenon may be administered in a subanesthetic dose to a sickle cell patient.

Xenon was first described as an anesthetic agent in the 1940s and is currently approved in several countries outside the U.S (e.g. Germany, France, Russia) as a general anesthetic. This shows a long-standing safety record for human use. Xenon has many properties of an ideal anesthetic agent. When xenon is inhaled at fractional concentrations of 0.63-0.73 with oxygen (1 MAC in human adults), induction of and emergence from anesthesia is rapid due to its low blood-gas partition coefficient (0.115). Anesthesia with xenon produces remarkable cardiovascular stability, attenuates hemodynamic responses to incision, exerts a narcotic-sparing effect, and demonstrates a remarkable ability to protect the brain and the heart against ischemic injury. It is noteworthy that xenon achieves some of these organ/tissue protective effects at inhaled concentrations that are much lower than those required for general anesthesia (i.e. 10-20%) and therefore fall well within the range of doses suggested here for the treatment of painful crisis.

In another embodiment xenon may be administered to a sickle cell patient undergoing surgery. Sickle cell patients are at increased risk to experience sickle cell related complications in the peri-operative period. An increased incidence of acute painful crisis, acute chest syndrome, and cerebrovascular accident has been described. Although the mechanisms of this increased perioperative risk are not clear, hypoxia, intravascular volume depletion, and acidosis are commonly cited as contributory factors. Mostly due to the lack of informative studies, there are currently no recommendations that would guide the practicing anesthesiologist in the selection of a particular anesthetic technique or agent to anesthetize the surgical sickle cell patient. Since xenon binds to sickle hemoglobin and reduces aggregation during the period of surgical stress, it is better suited to modify perioperative risk than another anesthetic agent, which lacks this property. Based on the physical characteristics of xenon in comparison to the modern halogenated ether anesthetics isoflurane, sevoflurane, and desflurane, xenon demonstrates greater hemoglobin binding, faster association rates, and provides superior inhibition of polymer formation.

Due to the bioactivity of xenon as an inhibitor of sickle hemoglobin polymerization and its anti-nociceptive qualities, inhaled xenon at subanesthetic doses is an effective therapeutic modality for the treatment of acute painful crisis.

Although described above with reference to certain specific embodiments, the present invention is nevertheless not intended to be limited to the details shown. Rather, various modifications may be made in the details within the scope and range of equivalents of the claims and without departing from the spirit of the invention.

I claim:

1. A method for treating pain associated with hemoglobinopathy comprising administering to a patient in need thereof a therapeutically effective amount of xenon.

2. The method of claim 1 where the patient has sickle cell disease and is suffering from a pain crisis.

3. The method of claim 1 where xenon is the primary general anesthetic agent.

4. The method of claim 1 where the effective amount of xenon delivered is 10%-20% inhaled with oxygen.

5. The method of claim 1 where the effective amount of xenon delivered is 1%-30% inhaled with oxygen.

6. A method for treating a patient with sickle cell disease comprising administering inhaled xenon at subanesthetic doses as an effective therapeutic modality for the treatment of acute painful crisis.

7. A method for pain therapy comprising administering xenon to a sickle cell patient undergoing surgery.

8. The method of claim 7 where xenon is the primary general anesthetic agent.

9. The method of claim 7 where the amount of xenon delivered is 10%-20% inhaled with oxygen.

10. The method of claim 7 where the amount of xenon delivered is 1%-30% inhaled with oxygen.
